(19) United States
(12) Patent Application Publication
(10) Pub. No.: US 2023/0052649 A1
(43) Pub. Date: Feb. 16, 2023

(54) EXTERNAL DEVICE, BIOMETRIC INFORMATION MEASURING DEVICE, IMPLANT SENSOR AND IMPLANT DEVICE FOR MEASURING BIOMETRIC INFORMATION

(71) Applicant: SB Solutions Inc., Ulsan (KR)

(72) Inventors: Seungup Seo, Ulsan (KR); Namhwan Sung, Ulsan (KR); Hae Dong Lee, Ulsan (KR); Seong Mun Kim, Ulsan (KR); Ji Woong Song, Ulsan (KR); Jagannath Malik, Ulsan (KR)

(73) Assignee: SB Solutions Inc., Ulsan (KR)

(21) Appl. No.: 17/535,317

(22) Filed: Nov. 24, 2021

(30) Foreign Application Priority Data
Aug. 13, 2021 (KR) 10-2021-0107639

(51) Int. Cl. A61B 5/145; A61B 5/05; H01Q 7/00; H01Q 1/27; H01Q 1/38

(52) U.S. Cl. CPC A61B 5/14532; A61B 5/1451; A61B 5/05; H01Q 7/00; H01Q 1/273; H01Q 1/38

(57) ABSTRACT

Disclosed are a biometric information measuring apparatus and method. An external device according to an embodiment includes a dipole antenna and a cavity reflecting an electro-magnetic field, radiated by the dipole antenna, in a direction toward an inside of a body having a target analyte. The external device may be attached to the exterior of the body having the target analyte.

### CROSS-REFERENCE TO RELATED APPLICATION

This application is based on and claims priority under 35 U.S.C. 119 to Korean Patent Application No. 10-2021-0107639, filed on Aug. 13, 2021 in the Korean intellectual property office, the disclosures of which are herein incorporated by reference in their entireties.

### TECHNICAL FIELD

The following description relates to an external device, a biometric information measuring apparatus, an implant sensor and an implant device for measuring biometric information.

### BACKGROUND OF THE INVENTION

Cases in which adult-onset diseases, such as diabetes, hyperlipidemia and thrombosis, are increased continue to increase. Such diseases need to be periodically measured using various bio sensors because it is important to continuously monitor and manage the diseases. A common type of a bio sensor is a method of injecting, into a test strip, blood gathered from a finger and then quantizing an output signal by using an electrochemical method or a photometry method. Such an approach method causes a user a lot of pain because blood needs to be gathered every time.

For example, in order to manage diabetes of hundreds of millions of people worldwide, the most basic thing is to measure blood glucose. Accordingly, a blood glucose measuring device is an important diagnostic device inevitably necessary for a diabetic. Various blood glucose measuring devices are recently developed, but the most frequently used method is a method of gathering blood by pricking a finger and then directly measuring a concentration of glucose within the blood. An invasive test includes a method of measuring blood glucose through the recognition of an external reader after measuring the blood glucose for a given time by penetrating an invasive sensor into the skin.

In contrast, a non-invasive test includes a method using a light-emitting diode (LED)-photo diode (PD), etc. However, the non-invasive test has low accuracy due to an environmental factor, such as sweat or a temperature, an alien substance, etc. because the LED-PD is attached to the skin.

The aforementioned information is to merely help understanding, and may include contents which do not form a part of a conventional technology and may not include contents which may be presented to those skilled in the art through a conventional technology.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Embodiments of present disclosure provide an external device including a dipole antenna having a cavity.

Embodiments of present disclosure provide a biometric information measuring apparatus in which an external device including a dipole antenna having a cavity induces a current into a sensor loop of an implant device and the implant device may process the sensing of an analyte through the sensor loop into which the current has been induced.

Embodiments of present disclosure provide an implant sensor having a trapezoidal microstrip conducting wire.

Embodiments of present disclosure provide an implant device including an implant sensor having a trapezoidal microstrip conducting wire.

According to embodiments, there is provided a biometric information measuring apparatus including an implant device inserted into the body having a target analyte and configured to measure a signal reflected by a surrounding analyte by radiating an electromagnetic wave having a specific frequency, and at least one external device configured to supply power to the implant device and receive measurement data of the implant device.

According to an aspect, the implant device may perform measurement on the analyte diffused from a blood vessel of the target analyte to an interstitial fluid within a tissue. According to another aspect, the at least one external device may include a first external device and a second external device disposed at a given interval on the exterior of the body having the target analyte. The first external device and the second external device are coupled, and may measure an electromagnetic wave according to a change in the analyte concentration within the interstitial fluid on the outer surface of the skin of the target analyte. Calibration may be performed on a measured value of the analyte concentration by using measurement data measured by the implant device and electromagnetic waves measured by the first external device and the second external device.

According to still another aspect, the at least one external device may radiate an electromagnetic wave that reaches up to a depth of the blood vessel of the target analyte, and may perform measurement for the analyte by measuring a signal reflected by the analyte after reaching the blood vessel of the target analyte.

According to still another aspect, the implant device may include a package, a conductive via formed to connect the inside and outside of the package in at least some of the package, a measurement antenna connected to the conductive via outside the package, a pad formed within the package and having a system on chip (SOC) formed therein, and a conducting wire connecting the via and the pad.

According to still another aspect, the implant device may include a measurement antenna conducting wire disposed along the outermost area of the package of the implant device, and a power reception coil isolated from the measurement antenna conducting wire and disposed in a middle area of the package.

According to still another aspect, the implant device may include a power reception coil disposed along the outermost area of the package of the implant device, and a measurement antenna conducting wire isolated from the power reception coil and disposed in a middle area of the package.

According to still another aspect, the implant device may include a coil having a sensing function and a power reception function, and may switch the sensing function according to the reception of power from the at least one external device, the radiation of the electromagnetic wave and the measurement of a reflected signal by switching the sensing function and the power reception function.

According to still another aspect, the implant device may have the outside thereof coated with a material selected for the safety of a living body.

According to still another aspect, an inflammation suppressor may be applied or coated on the external casing of the implant device.

According to embodiments, there is provided a biometric information measuring method, including receiving, by an implant device inserted into the body having a target analyte, power from at least one external device disposed in the exterior of the body having the target analyte, radiating, by the implant device, an electromagnetic wave having a specific frequency by using the supplied power, measuring, by the implant device, a signal of the radiated electromagnetic wave reflected by a surrounding analyte, and transmitting, by the implant device, measurement data according to the measured signal to the at least one external device by using the supplied power.

According to embodiments, there is provided a biometric information measuring method, including supplying, by an external device disposed in the exterior of a target analyte, power to an implant device inserted into the body having the target analyte, receiving, by the external device, measurement data measured by the implant device by using the supplied power from the implant device, and calculating an analyte concentration based on the received measurement data.

There can be provided the external device including the dipole antenna having the cavity. There can be provided the biometric information measuring apparatus in which the external device including the dipole antenna having the cavity induces a current into the sensor loop of the implant device and the implant device can process the sensing of an analyte through the sensor loop into which the current has been induced.

There can be provided the implant sensor having the trapezoidal microstrip conducting wire.

There can be provided the implant device including the implant sensor having the trapezoidal microstrip conducting wire.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram illustrating an example of a biometric information measuring apparatus according to an embodiment of the present disclosure.

FIG. 2 is an exemplary diagram illustrating three modes of the biometric information measuring apparatus according to an embodiment of the present disclosure.

FIGS. 3 and 4 are diagrams illustrating an example of a sensor of an implant device according to an embodiment of the present disclosure.

FIGS. 5 to 7 are diagrams illustrating another example of a sensor of the implant device according to an embodiment of the present disclosure.

FIG. 8 is a graph illustrating the results of simulations of sensors in embodiments of the present disclosure.

FIG. 9 is a diagram illustrating an example of a dipole antenna.

FIG. 10 is a diagram illustrating an example in which the directivity of the dipole antenna was improved using a cavity in an embodiment of the present disclosure.

FIG. 11 is a graph illustrating an example of a comparison between a dipole antenna and a dipole antenna having a cavity.

FIG. 12 is a diagram illustrating a dipole antenna having a cavity, of the external device, and a sensor loop of the implant device in an embodiment of the present disclosure.

FIG. 13 is a graph illustrating performance according to an in-vitro sensor of an external device in an embodiment of the present disclosure.

FIG. 14 is a graph illustrating an example of moment of multipole expansion of MLMA in an embodiment of the present disclosure.

FIG. 15 is a graph illustrating an example of moment of the dipole antenna having the cavity which interacts with multipole expansion of MLMA in an embodiment of the present disclosure.

FIG. 16 illustrates an example of a distribution of charges formed in a loop in an embodiment of the present disclosure.

### DETAILED DESCRIPTION

While illustrative embodiments have been illustrated and described, it will be appreciated that various changes can be made therein without departing from the spirit and scope of the invention.

Hereinafter, embodiments are described in detail with reference to the accompanying drawings. However, the embodiments may be changed in various ways, and the scope of right of this patent application is not limited or restricted by such embodiments. It is to be understood that all changes, equivalents and substitutions of the embodiments are included in the claims of the application.

Terms used in embodiments are merely used for a description purpose and should not be interpreted as intending to restrict the present disclosure. An expression of the singular number includes an expression of the plural number unless clearly defined otherwise in the context. In this specification, it should be understood that a term, such as “include” or “have”, is intended to designate the presence of a characteristic, a number, a step, an operation, a component, a part or a combination of them described in the specification, and does not exclude the existence or possible addition of one or more other characteristics, numbers, steps, operations, components, parts, or combinations of them in advance.

All terms used herein, including technical or scientific terms, have the same meanings as those commonly understood by a person having ordinary knowledge in the art to which an embodiment pertains, unless defined otherwise in the specification. Terms, such as those commonly used and defined in dictionaries, should be construed as having the same meanings as those in the context of a related technology, and are not construed as being ideal or excessively formal unless explicitly defined otherwise in the specification.

Furthermore, in describing the present disclosure with reference to the accompanying drawings, the same component is assigned the same reference numeral regardless of its reference numeral, and a redundant description thereof is omitted. In describing an embodiment, a detailed description of a related known art will be omitted if it is deemed to make the subject matter of the embodiment unnecessarily vague.

Furthermore, in describing elements of an embodiment, terms, such as a first, a second, A, B, (a), and (b), may be used. Such terms are used only to distinguish one component from the other component, and the essence, order, or sequence of a corresponding component is not limited by the terms. When it is said that one component is “connected”, “combined”, or “coupled” to the other component, the one component may be directly connected or coupled to the other component, but it should also be understood that a third component may be “connected”, “combined”, or “coupled” between the two components.

A component included in any one embodiment and a component including a common function are described using the same name in another embodiment. Unless described otherwise, a description written in any one embodiment may be applied to another embodiment, and a detailed description in a redundant range is omitted.

According to an embodiment, there is provided a technique relating to an in-body bio sensor capable of semi-permanently measuring blood glucose. The in-body bio sensor may also be called an invasive type bio sensor, an insertion type bio sensor, or an implant type bio sensor.

The in-body bio sensor may be a sensor for sensing a target analyte by using an electromagnetic wave. For example, the in-body bio sensor may measure biometric information associated with a target analyte. Hereinafter, the target analyte is a material associated with a living body, and may also be called a living body material or an analyte. For reference, in this specification, the target analyte is chiefly described as blood glucose, but the present disclosure is not limited thereto. The biometric information is information related to a bio component of a target, and may include a concentration of a target analyte or a numerical value, for example. If a target analyte is blood glucose, biometric information may include a blood glucose numerical value.

The in-body bio sensor may measure a bio parameter (hereinafter referred to as a “parameter”) associated with a bio component, and may determine biometric information from the measured parameter. In this specification, a parameter may indicate a circuit network parameter used to interpret a bio sensor and/or a bio sensing system, and is described by chiefly taking a scattering parameter as an example, for convenience of description, but the present disclosure is not limited thereto. For example, an admittance parameter, an impedance parameter, a hybrid parameter, a transmission parameter, etc. may be used as the parameter. A permeability coefficient and a reflection coefficient may be used as the scattering parameter. For reference, a resonant frequency calculated from a scattering parameter may be related to a concentration of a target analyte. The in-body bio sensor may predict blood glucose by sensing a change in the permeability coefficient and/or the reflection coefficient.

The in-body bio sensor may include a resonator assembly (e.g., an antenna). Hereinafter, an example in which the resonator assembly is an antenna is chiefly described. A resonant frequency of an antenna may be represented as a capacitance component and an inductance component as in Equation 1.

$$f = \frac{1}{2 π \sqrt{LC}} \qquad [ Equation 1 ]$$

In Equation 1, ƒ may indicate the resonant frequency of the antenna included in the in-body bio sensor using an electromagnetic wave. L may indicate inductance of the antenna. C may indicate capacitance of the antenna. The capacitance C of the antenna may be proportional to a relative dielectric constant εᵣ as in Equation 2 below.

C∝εrᵣ [Equation 2]

The relative dielectric constant εᵣ of the antenna may be influenced by a concentration of a surrounding target analyte. For example, when an electromagnetic wave passes through a material having a given dielectric constant, amplitude and a phase of the electromagnetic wave may be changed due to the reflection and scattering of the electromagnetic wave. The relative dielectric constant εᵣ may also vary because a degree of the reflection and/or scattering of the electromagnetic wave is different depending on a concentration of a target analyte around the in-body bio sensor. It may be interpreted that bio capacitance is formed between the in-body bio sensor and the target analyte due to a fringing field attributable to the electromagnetic wave radiated by the in-body bio sensor including an antenna. A resonant frequency of the antenna also varies because the relative dielectric constant εᵣ of the antenna varies depending on a change in the analyte concentration. In other words, the analyte concentration may correspond to the resonant frequency.

The in-body bio sensor according to an embodiment may radiate an electromagnetic wave while sweeping a frequency, and may measure a scattering parameter according to the radiated electromagnetic wave. The in-body bio sensor may determine a resonant frequency from the measured scattering parameter, and may estimate a blood glucose numerical value corresponding to the determined resonant frequency. The in-body bio sensor may be inserted into a subcutaneous layer, and may predict blood glucose diffused from a blood vessel to an interstitial fluid.

The in-body bio sensor may estimate biometric information by determining a frequency shift degree of a resonant frequency. In order to more accurately measure the resonant frequency, a quality factor may be maximized. Hereinafter, an antenna structure having an improved quality factor in an antenna device used in a bio sensor using an electromagnetic wave is described.

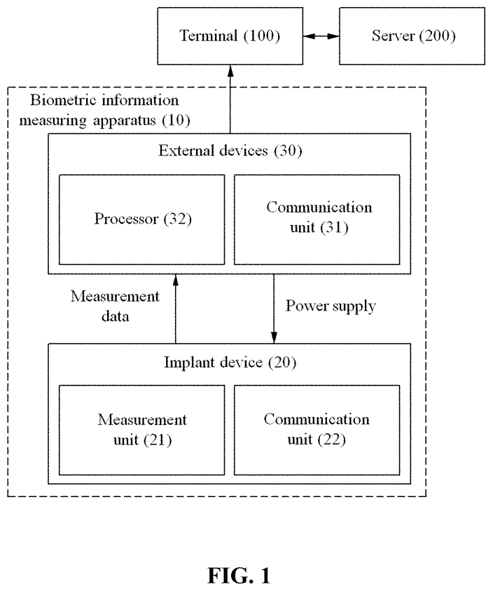

FIG. 1 is a block diagram illustrating an example of a biometric information measuring apparatus according to an embodiment of the present disclosure. The biometric information measuring apparatus 10 according to the present embodiment may include an implant device 20 inserted into the body having a target analyte whose biometric information (e.g., an analyte concentration, such as blood glucose or oxygen saturation) is to be measured and an external devices 30 disposed in the exterior of the target analyte at a location corresponding to a location of the implant device 20. The target analyte may be a human being or an animal. In this case, the implant device 20 may correspond to the aforementioned in-body bio sensor.

The external device 30 is a sensor attached to the outside of the body having the target analyte or worn by the target analyte, and may be fixed to the exterior of the target analyte by using various methods, such as a bending method and an adhesive method. The external device 30 may include a communication unit 31, and the external devices 30 may be paired through the communication units 31 or may provide biometric information to a preset terminal 100.

According to an embodiment, the external device 30 may also provide biometric information itself to the terminal 100, may perform a variety of types of analysis on the biometric information, and may provide the terminal 100 with the results of the analysis, a warning, etc. If the external device 30 provides biometric information itself to the terminal 100, the terminal 100 may perform a variety of types of analysis on the biometric information. Means for analyzing such biometric information may be easily selected by a practicer.

Furthermore, the external devices 30 can secure measurement accuracy and measurement continuity by blocking a performance change attributable to an external environment. The external devices 30 can improve accuracy by securing complementary data with the implant device 20.

The implant device 20 may be inserted into the body having a target analyte. For example, the implant device 20 does not directly come into contact with blood or is not disposed within a blood vessel, but may be disposed in an area other than a blood vessel at a given depth from the skin of a target. In other words, the implant device 20 is preferably disposed in a hypodermic area between the skin and the blood vessel.

The implant device 20 may radiate an electromagnetic wave having a specific frequency, and may measure a concentration of an analyte by measuring a signal reflected by the analyte around a sensor. For example, if blood glucose is to be measured, the implant device 20 may radiate an electromagnetic wave having a specific frequency, and may measure biometric information, such as blood glucose, by measuring a signal reflected by an analyte, such as glucose around a sensor.

The external devices 30 may be disposed in the exterior of a target analyte at a location corresponding to a location where the implant device 20 is disposed, supply power to the implant device 20, and may receive measurement data (e.g., the aforementioned biometric information) measured by the implant device 20.

When a concentration (e.g., a blood glucose numerical value) of a target analyte within a blood vessel of the target analyte is changed, a concentration of the analyte in a hypodermic area may be changed. In this case, a dielectric constant in the hypodermic area may be changed in response to a change in the concentration of the analyte. At this time, a resonant frequency in a measurement unit 21 of the implant device 20 may be changed in response to a change in the dielectric constant of a surrounding hypodermic area. For example, the measurement unit 21 may include a conducting wire having a specific pattern and a feeder line. In this case, when the dielectric constant of the surrounding hypodermic area is changed, a resonant frequency attributable to the specific pattern and the feeder line may also be changed because capacitance of the measurement unit 21 is changed. An analyte concentration under the skin is changed in proportion to an analyte concentration of a neighbor blood vessel. Accordingly, the biometric information measuring apparatus 10 may finally calculate biometric information, such as an analyte concentration, by using a resonant frequency corresponding to a change in the dielectric constant under the skin.

As an embodiment, the biometric information measuring apparatus 10 may calculate a corresponding relative dielectric constant by using a frequency (e.g., a resonant frequency) at a point at which the size of a scattering parameter is the smallest or greatest.

As an embodiment, the measurement unit 21 of the implant device 20 may be constructed in the form of a resonant device. The implant device 20 may generate a signal by sweeping a frequency within a pre-designated frequency band and inject the generated signal into the resonant device. At this time, the external devices 30 may measure a scattering parameter with respect to the resonant device to which a signal having a varying resonant frequency is supplied.

A communication unit 22 of the implant device 20 may transmit, to the external devices 30, data measured by the measurement unit 21. The communication unit 22 may receive, from the external device 30, power for generating a signal supplied to the measurement unit 21 by using a wireless power transmission method.

The external devices 30 may include a processor 32 and the communication unit 31. The communication unit 31 may receive measurement data (e.g., a scattering parameter or a degree of a change in the resonant frequency) measured by the implant device 20. In this case, the processor 32 of the external devices 30 may determine an analyte concentration based on the measurement data received from the implant device 20. According to an embodiment, the analyte concentration may be directly determined by the external devices 30, but may be determined by the terminal 100 that receives the measurement data from the external devices 30.

As an embodiment, a lookup table (LUT) in which measurement data (e.g., a scattering parameter and/or a degree of a change in the resonant frequency) and analyte concentrations are previously mapped may be stored in the external devices 30. The processor 32 may load an analyte concentration based on the LUT.

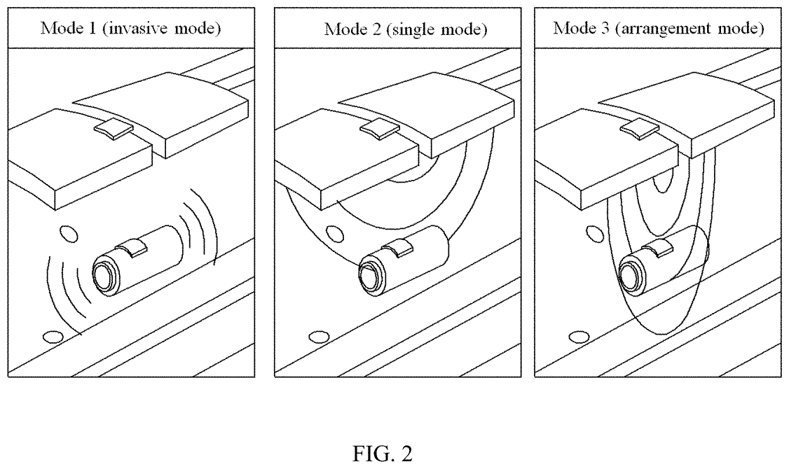

FIG. 2 is an exemplary diagram illustrating three modes of the biometric information measuring apparatus according to an embodiment of the present disclosure. The biometric information measuring apparatus 10 according to the present embodiment may operate in the three modes. The three modes may be independently performed or may be alternately performed at given time intervals.

<Mode 1: Invasive Mode>

In Mode 1, the biometric information measuring apparatus 10 may directly measure an analyte diffused from a blood vessel of a target to an interstitial fluid within a tissue. For example, an IC chip as the measurement unit 21 of the implant device 20 may radiate an electromagnetic wave having a specific frequency to an analyte, such as glucose around the implant device 20, and may measure a signal reflected and returned from the analyte. Furthermore, the implant device 20 may output a waveform (e.g., a sine wave) of a resonant frequency that varies over time. When a reflection signal according to the frequency in a specific time is detected, the implant device 20 may generate measurement data for biometric information corresponding to the frequency.

<Mode 2: Single Mode>

In Mode 2, at least one external device 30 is provided. Preferably, two external devices 30 may be provided. In this case, in Mode 2, the external devices 30 may include a first external device and a second external device disposed at a given interval.

The biometric information measuring apparatus 10 is coupled to the first external device and the second external device disposed at a given intervals, may measure an electromagnetic wave according to a change in an analyte concentration within an interstitial fluid on the outer surface of the skin of a target analyte, and may calibrate a measured value based on measurement data of biometric information of the implant device 20 along with the measured electromagnetic wave. The biometric information measuring apparatus 10 can improve the accuracy of measurement of an analyte concentration by calibrating a measured value through such a multi-mode.

<Mode 3: Arrangement Mode>

In Mode 1, the implant device 20 of the biometric information measuring apparatus 10 radiates an electromagnetic wave to an analyte around the implant device 20 and measures a signal reflected and returned from the analyte.

In contrast, in Mode 3, the biometric information measuring apparatus 10 radiates an electromagnetic wave that reaches even a depth of a blood vessel of a target analyte, and generates measurement data for biometric information (as an analyte concentration, for example, a blood glucose numerical value) based on a signal reflected and returned from the analyte within the blood vessel.

In general, when an analyte concentration within a blood vessel is changed, the analyte concentration in the hypodermic area may be changed. A dielectric constant in the hypodermic area is changed in response to a change in the analyte concentration.

In Modes 1 and 2, measurement data for biometric information is generated by performing measurement on such a hypodermic area. For this reason, there may be a difference between an analyte concentration within an actual blood vessel and an analyte concentration within a hypodermic area.

Accordingly, the biometric information measuring apparatus 10 can solve a time delay problem with an analyte concentration in a way to obtain measurement data for biometric information within an actual blood vessel by executing an operation, such as Mode 3. Furthermore, a problem which may occur in a target analyte during the time delay can be rapidly checked in advance because a sudden change in the analyte concentration of the target analyte can be measured by Mode 3.

Furthermore, the biometric information measuring apparatus 10 according to an embodiment can measure biometric information more accurately in a way to calibrate a value of an analyte concentration by simultaneously operating two or more of the three modes. For example, the biometric information measuring apparatus 10 according to the present embodiment can secure accuracy in a way to secure the diversity of data by simultaneously using Modes 1 and 2 of the implant device 20, and can improve the accuracy of measurement of biometric information through a repetition test.

Furthermore, the biometric information measuring apparatus 10 according to an embodiment can solve the time delay problem, that is, a problem with conventional interstitial fluid sensors for measuring biometric information, by improving a penetration depth of an electromagnetic wave radiated using Mode 3 and monitoring a change in the analyte concentration within a blood vessel in real time.

Furthermore, as described above, the diversity of data can be secured by using a plurality of sensors and a plurality of modes together. The accuracy of a method of predicting biometric information can be improved and a reappearance issue can be solved by adjusting a calibration cycle.

As an embodiment, the biometric information measuring apparatus 10 may predict an analyte concentration by associating, with a Bayesian filter-based algorithm, measurement data of another sensor (e.g., an environment sensor, a temperature sensor or a humidity sensor) along with measurement data measured by Modes 1, 2, and 3.

Furthermore, the biometric information measuring apparatus 10 according to an embodiment may simultaneously use Modes 1 and 2 in order to secure the reappearance of measurement of a dielectric constant, and may perform re-measurement when analyte concentrations measured based on measurement data of Mode 1 and measurement data of Mode 2 are not the same, or may measure an analyte concentration through blood-gathering, may input the measured analyte concentration, and may perform calibration.

As described above, the biometric information measuring apparatus 10 may perform mutual verification on the results of measurement when values of analyte concentrations measured in multiple modes are the same based on a plurality of measurement data obtained in the multiple modes, and may request to measure an analyte concentration of a target analyte through blood-gathering only when values of analyte concentrations measured in the multiples modes are different in order to reduce the number of times of blood-gathering for the target analyte.

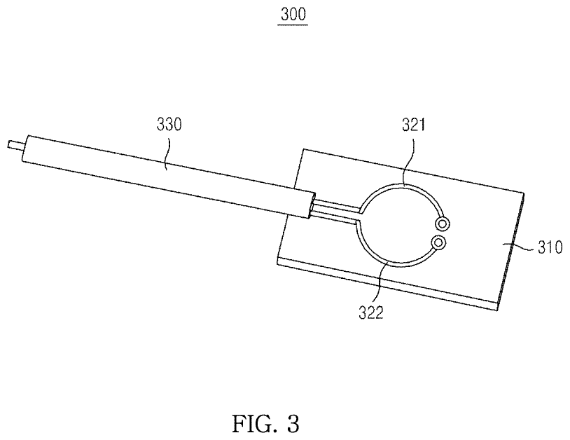

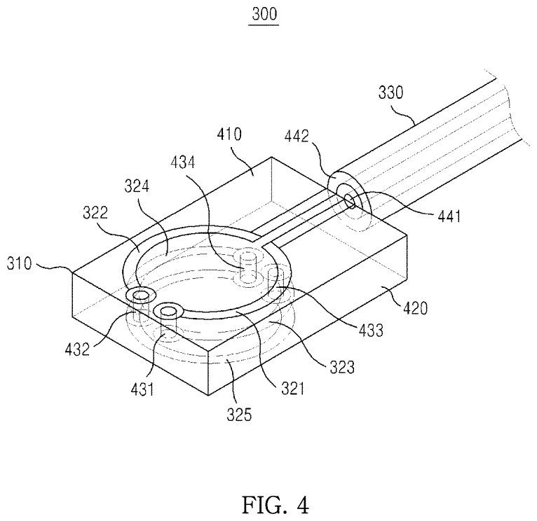

FIGS. 3 and 4 are diagrams illustrating an example of a sensor of the implant device according to an embodiment of the present disclosure. FIGS. 3 and 4 illustrate a sensor 300 corresponding to the measurement unit 21 of the implant device 20 according to the present embodiment. In this case, the sensor 300 may include conducting wires 321 to 325 having a specific pattern printed on a printed circuit board (PCB) 310 including multiple layers. A first conducting wire 321 and a second conducting wire 322 may be disposed in a first face 410 of the PCB 310. A fifth conducting wire 325 may be disposed in a second face 420 of the PCB 310. Finally, a third conducting wire 323 and a fourth conducting wire 324 may be disposed in a third face between the first face 410 and the second face 420. The faces in which the conducting wires 321 to 325 are disposed may be composed of layers. In this case, the first conducting wire 321 may be connected to the third conducting wire 323 through a first connection part 431. The second conducting wire 322 may be connected to the fourth conducting wire 324 through a second connection part 432. Furthermore, the third conducting wire 323 may be connected to the fifth conducting wire 325 through a third connection part 433. The fourth conducting wire 324 may be connected to the fifth conducting wire 325 through a fourth connection part 434. In this case, the connection parts 431 to 434 may connect the conducting wires 321 to 325 through via holes.

Furthermore, the first conducting wire 321 and the second conducting wire 322 may be connected to respective antenna ports. The antenna ports may be connected to a coaxial cable 330.

The coaxial cable 330 may include an inner conductor 441 and an outer conductor 442. For example, the first conducting wire 321 may be connected to the inner conductor 441. The second conducting wire 322 may be connected to the outer conductor 442. The coaxial cable 330 may supply a power source to the sensor 300 by using the inner conductor 441 and the outer conductor 442. For example, an end of the first conducting wire 321 connected to the inner conductor 441 may be an input port of the antenna port. An end of the second conducting wire 322 connected to the outer conductor 442 may be an output port of the antenna port.

In the sensor 300 according to the embodiment of FIGS. 3 and 4, a portion of a connection end with another system is not considered. Accordingly, there is proposed a sensor structure for compatibility (e.g., 50 ohm impedance matching) with another system and the easiness (soldering easiness) of supply of power.

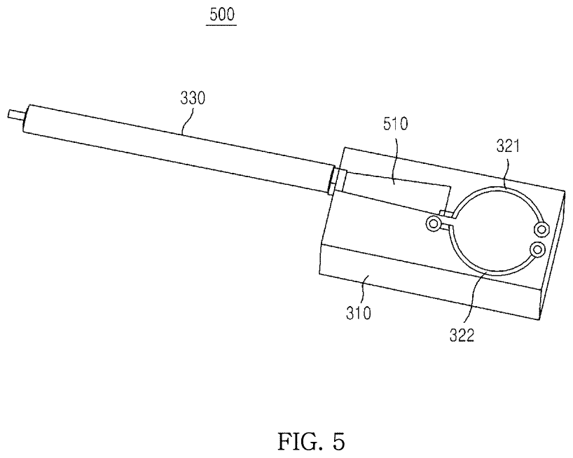

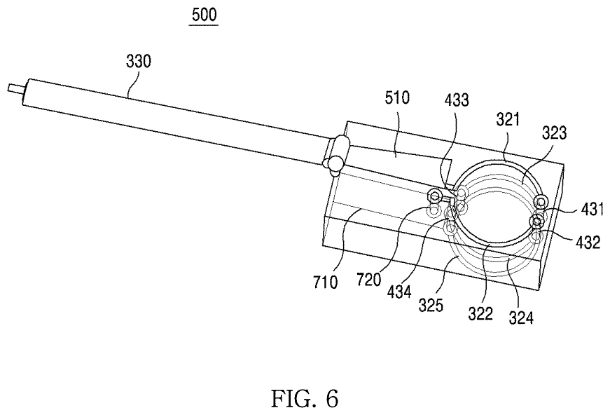

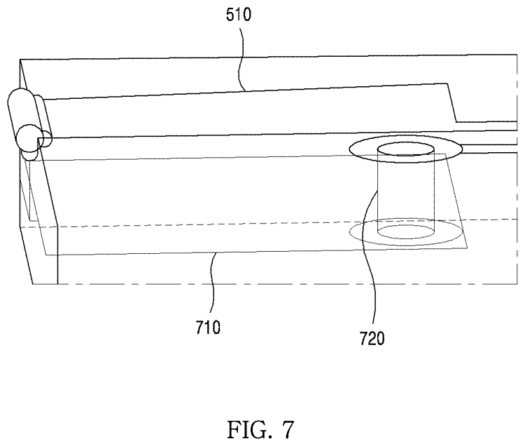

FIGS. 5 to 7 are diagrams illustrating another example of a sensor of the implant device according to an embodiment of the present disclosure. FIGS. 5 to 7 illustrate an example in which a sensor 500 according to an embodiment has a structure to which a microstrip conducting wire 510 has been added. In FIGS. 5 to 7, a structure connected to a coaxial cable for supplying a power source is omitted. Like the sensor 300 described with reference to FIGS. 3 and 4, the sensor 500 may include conducting wires 321 to 325 having a specific pattern printed on a PCB 310 including multiple layers. In this case, the microstrip conducting wire 510 is an added transmission wire that connects the first conducting wire 321 and a coaxial cable 330, and may minimize a power loss by converting input impedance of a multi-loop portion formed by the conducting wires 321 to 325 of the sensor 500 and impedance of a feeding system. Furthermore, the sensor 500 can be applied more widely because a connection end with another system is added through the microstrip conducting wire 510.

In the sensor 500, the second conducting wire 322 is not connected to the coaxial cable 330, and may be connected, through a fifth connection part 720, to a conductor 710 as a ground line having a quadrangle shape formed in an internal surface (e.g., a third face in which the third conducting wire 323 and the fourth conducting wire 324 are formed) of the PCB 310. In this case, the microstrip conducting wire 510 and the conductor 710 may be formed to face each other in parallel, and may deliver energy with waves confined therein between the microstrip conducting wire 510 and the conductor 710.

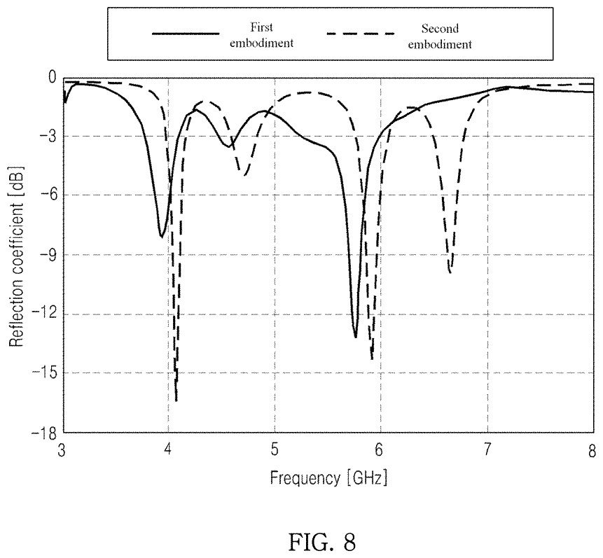

FIG. 8 is a graph illustrating the results of simulations of sensors in embodiments of the present disclosure. In this case, a first embodiment illustrates the results of simulations when sensing is performed using the sensor 300 according to the embodiment of FIGS. 3 and 4. A second embodiment illustrates the results of simulations when sensing is performed using the sensor 500 according to the embodiment of FIGS. 5 to 7. From the graph of FIG. 8, it may be seen that resonances necessary to measure an analyte are the same in both the embodiments. Results similar to the results of the simulations may be obtained even in results of measurement according to an actual implementation.

Embodiments in which the implant device 20 supplies power to the sensors 300 and 500 by using self-power have been described in relation to the sensors 300 and 500 described with reference to FIGS. 3 to 7. In another embodiment, the external devices 30 may transmit energy to the sensor of the implant device 20 by using a dipole antenna having a cavity.

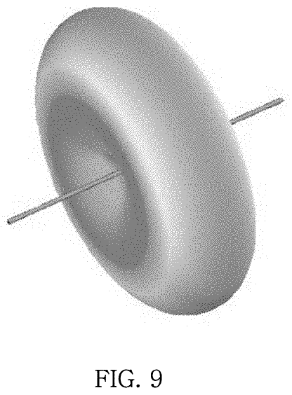

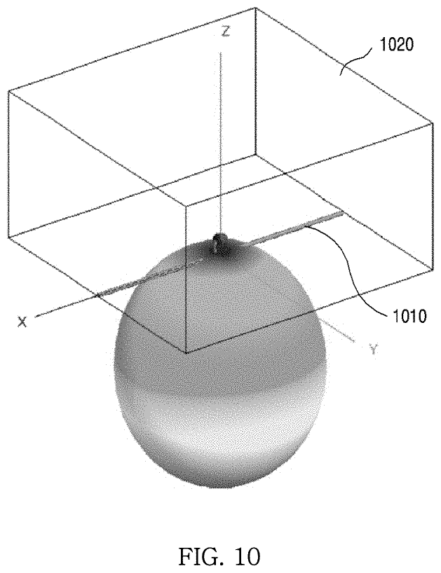

FIG. 9 is a diagram illustrating an example of a dipole antenna. FIG. 10 is a diagram illustrating an example in which the directivity of the dipole antenna was improved using a cavity in an embodiment of the present disclosure.

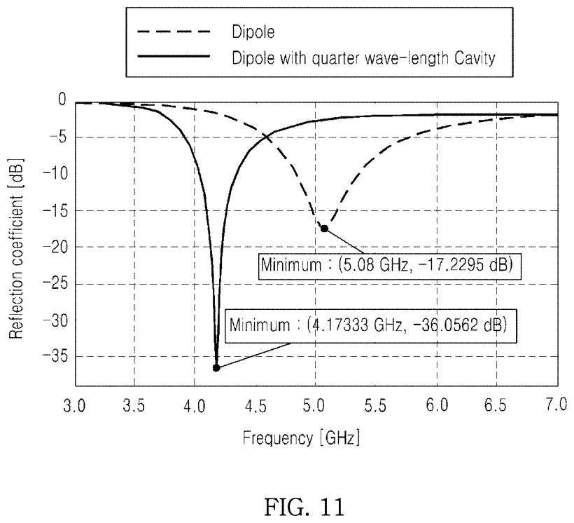

The dipole antenna or a doublet antenna illustrated in FIG. 9 is an antenna in which two straight-line conducting wires (elements) are attached to a cable end (feeding point) in a bilateral symmetry way. The dipole antenna is an antenna, that is, a basis of a line-shaped antenna, along with a monopole antenna, and is an antenna having the simplest structure. In FIG. 10, a cavity 1020 is disposed on one side of a dipole antenna 1010. The directivity of a electro-magnetic field radiated by the dipole antenna 1010 can be improved through the cavity 1020. In order to improve the directivity by using the cavity 1020, a Febry-Perot method may be used. A pattern radiated by the dipole antenna 1010 may include a first field (e.g., the upper side of FIG. 9) proceeding toward the cavity 1020 and a second field (e.g., the lower side of FIG. 9) proceeding in a direction opposite the cavity 1020. In this case, the first field is reflected by the cavity 1020 made of a metal conductor. At this time, the phase of the first field may be changed by 180 degrees. If the depth of the cavity 1020 is designed to have a quarter wavelength, a phase shift of 90 degrees from the dipole antenna 1010 to the conductor of the cavity 1020 may occur. If a round-trip is considered, a total of a phase shift of 360 degrees, including a phase shift of 90 degrees from the dipole antenna 1010 to the cavity 1020, a phase shift of 180 degrees attributable to reflection, and a phase shift of 90 degrees from the cavity 1020 to the dipole antenna 1010, may occur. Accordingly, a field in which the second field (0 degree) and the first field reflected and returned from the cavity 1020 are co-phased and overlapped may be doubled in the direction opposite to the cavity 1020. FIG. 11 is a graph illustrating an example of a comparison between a common dipole antenna and the dipole antenna 1010 having the cavity 1020. As illustrated in the graph of FIG. 11, it may be seen that the dipole antenna 1010 having the cavity 1020 has a lower reflection coefficient in a lower frequency compared to the common dipole antenna.

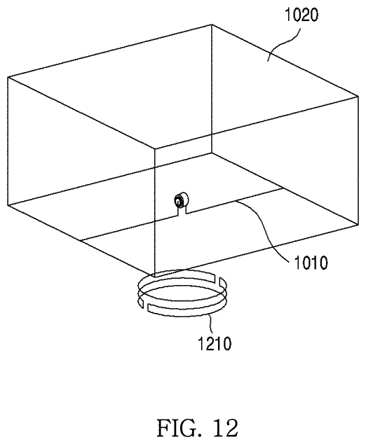

FIG. 12 is a diagram illustrating a dipole antenna having a cavity, of the external device, and a sensor loop of the implant device in an embodiment of the present disclosure. A electro-magnetic field generated by the electric dipole of the dipole antenna 1010 having the cavity 1020, of the external devices 30 disposed in the exterior of a target analyte, that is, by the dipole antenna 1010, may induce a current into a sensor loop 1210 through an interaction with the sensor loop 1210 of the implant device 20 inserted into the body having the target analyte. In other words, although the implant device 20 does not have a self-power source, a current can be induced into the sensor loop 1210, and also sensing can be performed using the sensor loop 1210 of the implant device 20 through the induced current. In this case, the dipole antenna 1010 may radiate a electro-magnetic field into the body having the target analyte twice as hard due to the deployment of the cavity 1020. In other words, a field radiated to the outside of a body can be minimized. If a field is radiated to the outside of the body, for example, when a hand of a person having a high dielectric constant passes by the dipole antenna 1010 or nearby, a sensing characteristic may be distorted because a surrounding field is distorted. Accordingly, the deployment of the cavity 1020 can maximize in-body radiation, and can minimize the distortion of a characteristic attributable to an environment change outside the body because the deployment of the cavity 1020 is used as a scheme for minimizing radiation to the outside of the body.

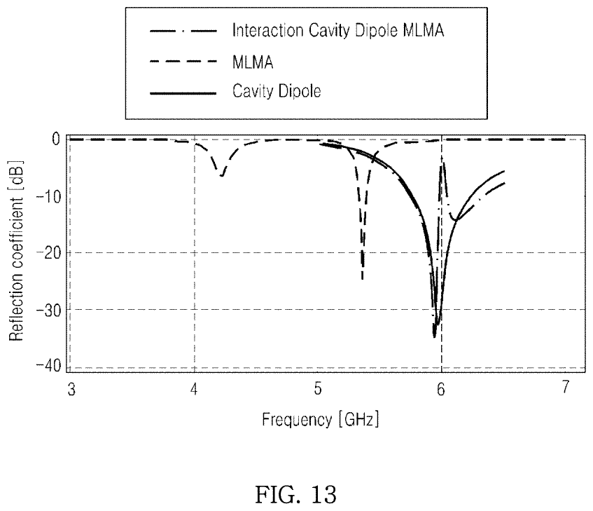

FIG. 13 is a graph illustrating performance according to an in-vitro sensor of an external device in an embodiment of the present disclosure. FIG. 13 is a graph for describing performance of powerless sensing. If the dipole antenna outside a body and the loop antenna within the body are coupled, information on an analyte within the body may be sensed by monitoring a change in a corresponding peak frequency because a resonant peak occurs due to a dark mode (i.e., a phenomenon in which an energy trap occurs within dipole resonance). A power source is connected to the dipole antenna. A loop sensor mounted on the implant device may sense the analyte because a peak frequency in a trapped mode occurring due to external dipole resonance is shifted due to a change in a concentration of the internal analyte (e.g., a change in blood glucose) although a power source is not connected to the loop sensor. FIG. 13 illustrates performance of a microstrip line monopole antenna (MLMA) and a cavity dipole antenna. It may be seen that the cavity dipole antenna has a lower reflection coefficient.

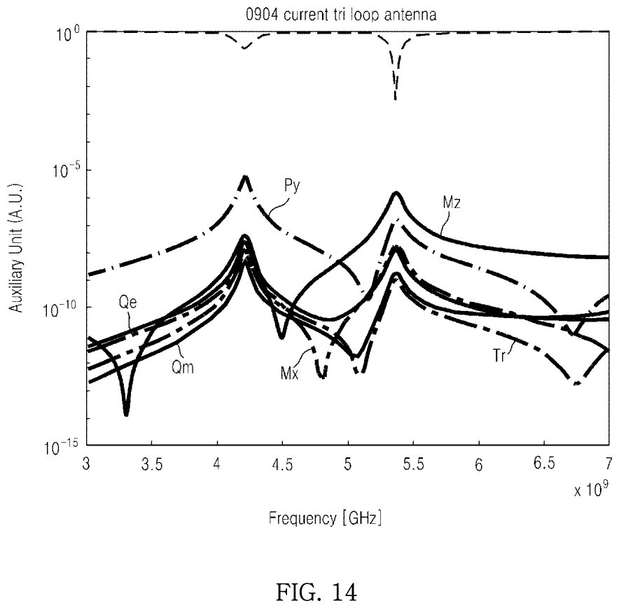

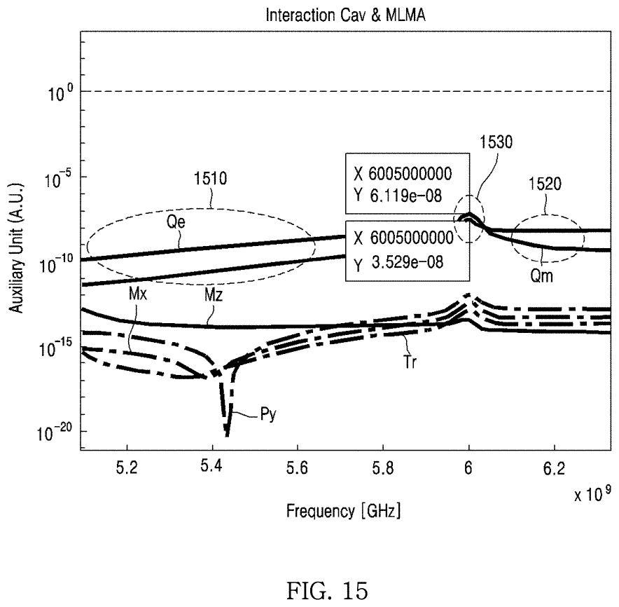

FIG. 14 is a graph illustrating an example of moments of multipole expansion of the MLMA in an embodiment of the present disclosure. FIG. 15 is a graph illustrating an example of moments of the cavity dipole antenna which interacts with the multipole expansion of the MLMA in an embodiment of the present disclosure. In the graph of FIG. 14 and the graph of FIG. 15, a transverse axis (x-axis) indicates a frequency, and a longitudinal axis (y-axis) indicates an auxiliary unit (a.u.). Furthermore, in the graph of FIG. 14, a dotted line indicates a reflection coefficient. In the graph of FIG. 15, a dotted line indicates a reference line. P, M, and T may mean moments of an electric dipole, a magnetic dipole, and a toroidal dipole, respectively. Since each of the moments has a vector quantity, x, y, and z may mean three-dimensional vector components. Qₑ may mean an electric quadrupole, and Qₘ may mean a magnetic quadrupole. If a mathematical transform called the multipole expansion is used, a current density applied to metal of the sensor may be represented as the overlap of multipole moments. Each multipole moment is an orthogonal component. In this case, the graphs of FIGS. 14 and 15 may be graphs drawn by diagramming a level of contribution of each of the moments with respect to the frequency. As described above, a dominant moment component in each of the frequencies through the graphs of FIGS. 14 and 15.

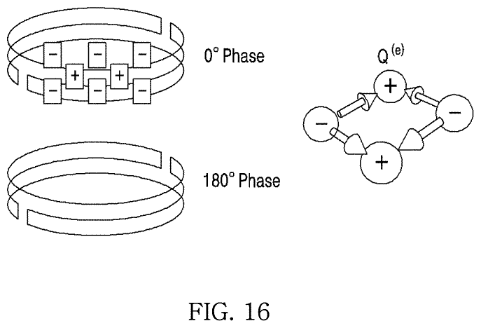

The graph of FIG. 14 illustrates that the magnetic dipole P is dominant at a resonant point. The graph of FIG. 15 illustrates that the electric quadrupole (Qₑ) and the magnetic quadrupole (Qₘ) are dominant. In the trapped mode situation, it is shown that the magnetic quadrupole (Qₘ) is more dominant than the electric quadrupole (Qₑ). When a bright mode having a duplication behavior interacts with a sub-radiated dark mode, resonance in the trapped mode may occur. Such a trapped mode may have a very high Q coefficient due to a strong local field characteristic. In an electromagnetic area, short-range coupling may be used to excite the trapped mode. Furthermore, the graph of FIG. 15 illustrates that the electric quadrupole (Qₑ) has a more dominant frequency range than the magnetic quadrupole (Qₘ) in areas indicated by a first dotted circle 1510 and a second dotted circle 1520 and that the magnetic quadrupole (Qₘ) has a more dominant frequency range than the electric quadrupole (Qₑ) in an area indicated by a third dotted circle 1530. Furthermore, FIG. 16 illustrates an example of a distribution of charges formed in a loop in an embodiment of the present disclosure. The loop according to the embodiment of FIG. 16 has a top and bottom symmetrical shape, and may form sub-radiative resonance by inducing the offset of currents.

As described above, embodiments of the present disclosure can provide the external device including the dipole antenna having the cavity. Furthermore, embodiments of the present disclosure can provide the biometric information measuring apparatus in which the implant device can process the sensing of an analyte through the sensor loop into which a current has been induced by inducing the current into the sensor loop of the implant device through the external device including the dipole antenna having the cavity. Furthermore, embodiments of the present disclosure can provide the implant sensor having the trapezoidal microstrip conducting wire. Furthermore, embodiments of the present disclosure can provide the implant device including the implant sensor having the trapezoidal microstrip conducting wire.

The aforementioned system or device or apparatus may be implemented as a hardware component or a combination of a hardware component and a software component. For example, the device and component described in the embodiments may be implemented using a processor, a controller, an arithmetic logic unit (ALU), a digital signal processor, a microcomputer, a field programmable gate array (FPGA), a programmable logic unit (PLU), a microprocessor or one or more general-purpose computers or special-purpose computers, such as any other device capable of executing or responding to an instruction. The processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary skill in the art may understand that the processing device may include a plurality of processing elements and/or a plurality of types of processing elements. For example, the processing device may include a plurality of processors or a single processor and a single controller. Furthermore, a different processing configuration, such as a parallel processor, is also possible.

Software may include a computer program, a code, an instruction or a combination of one or more of them and may configure a processing device so that the processing device operates as desired or may instruct the processing devices independently or collectively. The software and/or the data may be embodied in any type of machine, a component, a physical device, a computer storage medium or a device in order to be interpreted by the processor or to provide an instruction or data to the processing device. The software may be distributed to computer systems connected over a network and may be stored or executed in a distributed manner. The software and the data may be stored in one or more computer-readable recording media.

The method according to embodiments may be implemented in the form of a program instruction executable by various computer means and stored in a computer-readable medium. The computer-readable medium may include a program instruction, a data file, and a data structure solely or in combination. The medium may continue to store a program executable by a computer or may temporarily store the program for execution or download. Furthermore, the medium may be various recording means or storage means having a form in which one or a plurality of pieces of hardware has been combined. The medium is not limited to a medium directly connected to a computer system, but may be one distributed over a network. An example of the medium may be one configured to store program instructions, including magnetic media such as a hard disk, a floppy disk and a magnetic tape, optical media such as a CD-ROM and a DVD, magneto-optical media such as a floptical disk, a ROM, a RAM, and a flash memory. Furthermore, other examples of the medium may include an app store in which apps are distributed, a site in which other various pieces of software are supplied or distributed, and recording media and/or storage media managed in a server. Examples of the program instruction may include machine-language code, such as a code written by a compiler, and a high-level language code executable by a computer using an interpreter.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned components, such as the system, configuration, device, and circuit, are coupled or combined in a form different from that of the described method or replaced or substituted with other components or equivalents.

Accordingly, other implementations, other embodiments, and the equivalents of the claims fall within the scope of the claims.

1. An external device comprising:
a dipole antenna configured to radiate a first electromagnetic field in a direction toward an outside of a body having the target analyte and a second electromagnetic field in a direction toward an inside of the body having the target analyte;
a processor configured to predict a future concentration of a target analyte by associating, with a Bayesian filter-based algorithm, humidity data of a humidity sensor, and an analyte concentration measured by an implant device; and
a cavity reflecting the first electro-magnetic field to generate a reflected electromagnetic field, wherein the cavity has a quarter wavelength depth,
wherein the reflected electromagnetic field and the second electromagnetic field are co-phased and overlapped in the direction towards the inside of the body;
wherein the external device is configured to be attached to an exterior of the body having the target analyte.

2. The external device of claim 1, wherein the cavity is made of a metal conductor.

3. The external device of claim 1, wherein a current is induced into a sensor loop of the implant device inserted into the body having the target analyte through the second electro-magnetic field radiated by the dipole antenna and the reflected electromagnetic field.

4. The external device of claim 3, wherein the implant device is configured to measure an analyte concentration within the body having the target analyte based on a third electro-magnetic field formed through the current induced into the sensor loop.

5. The biometric information measuring apparatus of claim 7, wherein the sensor loop comprises:
a first conducting wire and a second conducting wire isolated from each other and disposed along some of a boundary of a first area on a first plane;
a third conducting wire and a fourth conducting wire isolated from each other and disposed along some of a boundary of a second area on a second plane isolated from the first plane in parallel;
a fifth conducting wire disposed along some of a boundary of a third area on a third plane isolated from the second plane in parallel;
a first connection part connecting a first end of the first conducting wire and a first end of the third conducting wire;
a second connection part connecting a first end of the second conducting wire and a first end of the fourth conducting wire;
a third connection part connecting a second end of the third conducting wire and a first end of the fifth conducting wire; and
a fourth connection part connecting a second end of the fourth conducting wire and a second end of the fifth conducting wire.

6. The biometric information measuring apparatus of claim 5, wherein a second end of the first conducting wire is connected to an antenna port through a trapezoidal microstrip conducting wire.

7. A biometric information measuring apparatus comprising:
an external device configured to be attached to an exterior of a body having a target analyte and comprising:
a dipole antenna;
a first electro-magnetic field, radiated by the dipole antenna, in a direction toward an outside of the body having the target analyte;
a second electromagnetic field, radiated by the dipole antenna, in a direction towards an inside of the body; and
a cavity configured to reflect the first electromagnetic field to generate a reflected electromagnetic field, wherein the cavity has a quarter wavelength depth, and wherein the reflected electromagnetic field and the second electromagnetic field are co-phased and overlapped in the direction towards the inside of the body; and
an implant device configured to be inserted into the body having the target analyte, comprising a sensor loop, and configured to measure an analyte concentration within the body having the target analyte based on a third electro-magnetic field formed using a current induced into the sensor loop through the second electro-magnetic field radiated by the dipole antenna and the reflected electromagnetic field,
wherein the biometric information measuring apparatus is configured to predict a future analyte concentration by associating, with a Bayesian filter-based algorithm, humidity data from a humidity sensor, along with the analyte concentration of the implant device.

8. An implant sensor comprising: a sensor loop;
a trapezoidal microstrip conducting wire connected to one of a plurality of conducting wires constituting the sensor loop, and configured to measure a signal reflected by an analyte by radiating an implant electromagnetic wave having a frequency; and
a coil having a sensing function and a power reception function, wherein the coil is configured to switch the sensing function according to a reception of power from an external device, a second electromagnetic field, and a reflected electromagnetic field,
wherein a current is induced into the sensor loop of the implant device through the second electromagnetic field radiated by a dipole antenna and the reflected electromagnetic field, and wherein the reflected electromagnetic field and the second electromagnetic field are co-phased and overlapped in a direction towards an inside of a body.

9. The implant sensor of claim 8, wherein the plurality of conducting wires is printed on a printed circuit board (PCB) comprises of multiple layers.

10. The implant sensor of claim 8, further comprising a conducting wire disposed in a layer different from a layer of the conducting wire connected to the microstrip conducting wire among the plurality of conducting wires.

11. The implant sensor of claim 10, wherein the ground line has a quadrangle shape and is placed or oriented in parallel to the microstrip conducting wire in a printed circuit board (PCB) on which the plurality of conducting wires is printed.

12. The implant sensor of claim 8, wherein the sensor loop comprises:
a first conducting wire and a second conducting wire isolated from each other and disposed along some of a boundary of a first area on a first plane;
a third conducting wire and a fourth conducting wire isolated from each other and disposed along some of a boundary of a second area on a second plane isolated from the first plane in parallel;
a fifth conducting wire disposed along some of a boundary of a third area on a third plane isolated from the second plane in parallel;
a first connection part connecting a first end of the first conducting wire and a first end of the third conducting wire;
a second connection part connecting a first end of the second conducting wire and a first end of the fourth conducting wire;
a third connection part connecting a second end of the third conducting wire and a first end of the fifth conducting wire; and
a fourth connection part connecting a second end of the fourth conducting wire and a second end of the fifth conducting wire.

13. The implant sensor of claim 12, wherein the microstrip conducting wire connects a second end of the first conducting wire and an antenna port.

14. An implant device comprising the implant sensor according to claim 8 and inserted into a body having a target analyte.

15-16. (canceled)
